(19) United States
(12) Patent Application Publication
(10) Pub. No.: US 2021/0239717 A1
(43) Pub. Date: Aug. 5, 2021

(54) MICROSAMPLING DETECTION IN DIABETES

(71) Applicant: Quest Diagnostics Investments LLC, Secaucus, NJ (US)

(72) Inventors: Nigel Clarke, Vista, CA (US); Mildred Goldman, Laguna Niguel, CA (US); Jueun Kim, San Juan Capistrano, CA (US); Jane Yang, San Juan Capistrano, CA (US)

(73) Assignee: Quest Diagnostics Investments LLC, Secaucus, NJ (US)

(21) Appl. No.: 17/269,926

(22) PCT Filed: Aug. 21, 2019
(86) PCT No.: PCT/US2019/047572

§ 371 Date: Feb. 19, 2021

Related U.S. Application Data
(60) Provisional application No. 62/721,227, filed on Aug. 22, 2018.

(51) Int. Cl. G01N 33/72

(52) U.S. Cl. CPC G01N 33/723; H01J 49/0036; G01N 2800/7071; G01N 2440/38

(57) ABSTRACT

The present disclosure relates to methods of detecting various analytes, including glycated hemoglobin (HbA1c) and total hemoglobin (THb), in a biological sample obtained with a microsampling device.

### RELATED APPLICATIONS

This application claims priority under 35 U.S.C. § 119(e) to U.S. Provisional Application 62/721,227 filed Aug. 22, 2018, the entire contents of which are incorporated herein by reference.

### FIELD

The present disclosure relates generally to the field of detecting an analyte obtained from a microsampling device. Described are methods of detecting markers of diabetes, testosterone and/or other hormones, microalbumin, creatinine/estimated glomerular filtration rate (eGFR), thyroid stimulating hormone (TSH), C-reactive protein (CRP), vitamin D, and omega 3, as well as markers of kidney, liver, or thyroid function .

### BACKGROUND

The following discussion is provided to aid the reader in understanding the disclosure and is not admitted to describe or constitute prior art thereto.

Many current methods of diagnosis and biological sample analysis rely on fluid samples in relatively large quantities. Such samples require refrigeration in dry ice or freezing for transport, which is expensive and burdensome on personnel handling the samples. Also the fluid samples may be considered a biohazard that requires a special transport method, and the volume required to obtain the amount of fluid needed may require specialized personnel to be obtained, such as a phlebotomist for collecting blood by venipuncture. Alternative sample types are therefore of interest for improving patient experience and convenience.

Alternative sampling methods include using reduced sample volume, particularly for patients with frequent testing for several analytes (e.g., patients with anemia or diabetes). Methods that rely on reduced sample volume my utilize finger sticks to obtain a few drops of blood, as opposed to conventional phlebotomy, and this allows the process to proceed at home via self-collection by the patient. This reduces infrastructure costs and eases compliance for patient with difficult venous access (such as children and obese patients).

Accordingly, there is a need in the art to improve biological sampling and analysis of biological samples to improve patient compliance and ease of access. The present disclosure satisfies this need.

### SUMMARY

Described herein are devices and methods for detecting one or more analytes from a microsampling device used to collect a biological sample.

In one aspect, the present disclosure provides methods of determining a fraction of glycated hemoglobin (HbA1c) in a sample, comprising: eluting a biological sample from a microsampling device used to collect the biological sample an individual; extracting hemoglobin from the biological sample; measuring the concentration of HbA1c and the concentration of total hemoglobin (THb); calculating the fraction of HbA1c in the THb.

In some embodiments, the biological sample is obtained from an individual having or suspected of having diabetes.

In some embodiments, the biological sample is a dried fluid. In some embodiments, the biological sample is dried serum, dried capillary blood, or dried whole blood.

In some embodiments, the biological sample is collected from a patient via fingerstick. In some embodiments, the biological sample is eluted from an absorbent tip of the microsampling device by incubating the absorbent tip in water.

In some embodiments, extraction comprises contacting the biological sample with a lysis buffer to lyse erythrocytes in the sample. In some embodiments, the methods may further comprise contacting the sample with sodium nitrite. In some embodiments, the methods may further comprise contacting the sample with one or more of a protease, sodium azide, and fructosyl peptideoxidase.

In some embodiments, the concentration of THb is determined by measuring absorbance. In some embodiments, the concentration of HbA1c is determined by measuring an indirect marker. For example, in some embodiments, the indirect marker is hydrogen peroxide.

In some embodiments, the THb or the HbA1c are measured by mass spectroscopy, while in some embodiments, both the THb and the HbA1c are measured by mass spectroscopy.

In some embodiments, the microsampling device is a MITRA® tip. In some embodiments, the sample volume of the microsampling device is no more than about 10 to about 20 μL.

In some embodiments, the methods may further comprise detecting one or more of glucose, LDLp, creatinine, and microalbumin.

In some embodiments, the individual self-collects the biological sample. In some embodiments, the individual shipped the microsampling device used to self-collect the biological sample to a testing facility in a pre-addressed envelope.

In some embodiments, a fraction of HbA1c/THb that is 6.5% or higher is indicative of a lack of diabetic control.

In another aspect, the present disclosure provides methods of detecting an analyte in a sample, comprising: eluting a biological sample from a microsampling device used to collect the biological sample from an individual; extracting two or more analytes from the biological sample; measuring the concentration of two or more analytes; wherein the two or more analytes are selected from the group consisting of HbA1c, total hemoglobin, glucose, low density lipoprotein particle number (LDLp), microalbumin, creatinine/estimated glomerular filtration rate (eGFR), thyroid stimulating hormone (TSH), C-reactive protein (CRP), vitamin D, omega 3, a marker of kidney function (e.g., creatinine, urea, uric acid, electrolytes), a marker of liver function (e.g., liver transaminases aspartate transaminase, alanine transaminase, bilirubin, albumin, alkaline phosphatase, gamma glutamyl transpeptidase), and a marker of thyroid function (e.g., TSH, T4, T3).

The following detailed description is exemplary and explanatory, and is intended to provide further explanation of the invention.

### BRIEF DESCRIPTION OF THE DRAWINGS

FIG. 1 shows an exemplary microsampling device and its use in obtaining/storing blood from a finger-stick.

FIG. 2 shows a scatter plot of a Deming regression that validates the use of HbA1c/THb as an indicator of diabetic control (i.e., blood glucose levels over time).

FIG. 3 shows scatter plots of Deming regressions indicating that cortisol, testosterone, and progesterone are validated and detectable pursuant to the disclosed methods.

FIG. 4 shows a scatter plot of a Deming regression indicating that 25-Hydroxyvitamin D3 is validated and detectable pursuant to the disclosed methods.

### DETAILED DESCRIPTION

The methods disclosed herein provide a way to reduce cost and increase efficiency of biological sample processing while improving patient experience and compliance. The disclosed methods rely on the use of a microsampling device to obtain and store a biological sample (e.g., blood, plasma, saliva, urine, etc.) prior to processing and analyzing the sample.

In some aspects, the disclosed methods are particularly apt for diagnosing and monitoring diabetes (e.g., type 1, type 2, or gestational) by obtaining a microsample of a bodily fluid, eluting the biological sample from the microsampling device, extracting hemoglobin from the sample, and measuring the concentrations of glycated hemoglobin (HbA1c) and total hemoglobin (THb) in order to determine the fraction of HbA1c in THb. The higher the fraction of HbA1c to THb, the less controlled the diabetes. The disclosed methods allow for minimally invasive continuous monitoring that accurately reflects long-term glucose control, and therefore the methods described herein provide a considerable advance over conventional techniques.

Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, an “amount” of an analyte in a body fluid sample or culture refers generally to an absolute value reflecting the mass of the analyte detectable in the volume of sample or culture. However, an amount also contemplates a relative amount in comparison to another analyte amount. For example, an amount of an analyte in a sample can be an amount which is greater than a control or normal level of the analyte normally present in the sample.

As used herein, the terms “about” or “approximately” when preceding a numerical value indicates the value plus or minus a range of 10% of the value.

“Body fluid” and “bodily fluid,” used interchangeably herein, refer to a fluid sample from a human, animal, or cell culture. Body fluids include, but are not limited to amniotic fluid, blood, cerebrospinal fluid, peritoneal fluid, plasma, pleural fluid, saliva, semen, serum, sputum, tears, and urine. In preferred embodiments, the body fluid or bodily fluid is human plasma.

As used herein, the term “sample” refers to clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), or organ tissue (e.g., pancreatic tissue). Preferred sample sources include plasma, serum, or whole blood (dried or liquid).

“Individual,” “patient,” or “subject,” as used herein, can be an individual organism, a vertebrate, a mammal, or a human. In preferred embodiments, the individual, patient, or subject is a human.

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Microsampling Devices Employed in the Methods of the Present Technology

Conventional dried blood spotting techniques are accompanied by a number of drawbacks, including imprecise sample volume and reliance on a constant sample viscosity (i.e., the expectation that the sample will spread uniformly on the sample card). A constant viscosity results in blood spot diameters remaining constant when equal volume samples are administered to the cards. However, viscosity varies significantly between blood samples because of differing hematocrit (HCT) or packed cell volume (PCV) levels in the blood. Samples with high hematocrit levels form smaller diameter spots on the bloodspot papers, leading to different concentrations of blood within the fixed diameter of the spots sampled. PCV levels are believed to show a variance of about 45% in spot diameters. As internal standards are sprayed onto the spotted blood, this can result in a 45% error in quantitation. The microsampling devices employed in the methods disclosed herein confer several advantages, including the collection of more precise blood volumes, lack of hematocrit bias, and the ability to be easily automated with standard liquid handlers for lab processing.

Additionally, conventional blood spot techniques require a comparatively large volume of blood relative to the disclosed microsampling devices. A dried blood spot would generally require 50-75 μl per spot, while a microsampling device can yield results from approximately 20 μl. It has been recognized in the art that dried blood spots often have performance variability issues for detecting viral load compared to other samples types, such as plasma (Pannus et al., Medicine, 95:48(e5475) (2016)), and the volume of a dried blood spot may need to be significantly higher for certain types of assessment (e.g., optical density) compared to other sample types, such as serum (Brandao et al., J. Clin. Virol., 57:98-102 (2013)). Indeed, found that using both dried blood spot and plasma spot screening for detecting viral load and treatment failure in HIV patients receiving antiretroviral therapy found that both yielded a high rate of false positives (Sawadogo et al., J. Clin. Microbiol., 52(11):3878-83 (2014)).

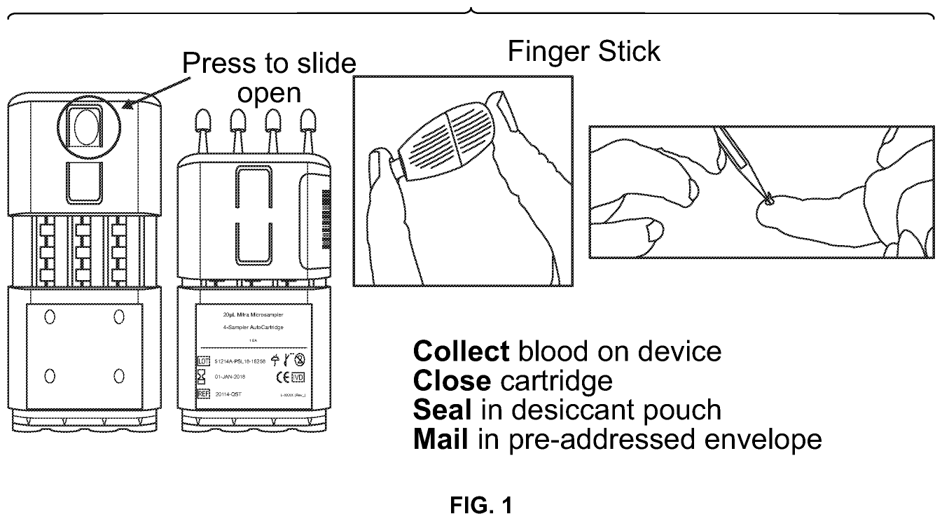

Microsampling devices useful in the methods of the present technology comprise an absorbent tip having a distal end and a proximal end. The width of the distal end of the absorbent tip is narrow compared to the width of the proximal end. The proximal end is attached to a holder, whereas the distal end is configured to contact a fluid to be absorbed, such as blood. The microsampling device permits biological fluid samples, such as blood, to be easily dried, shipped, and then later analyzed. In certain embodiments, the biological fluid is blood from a fingerstick. An exemplary microsampling device and its application to a finger stick is shown in FIG. 1, which further explains that in some embodiments the blood (or other biological sample) may be collected, sealed in a desiccant pouch, and mailed to a laboratory for analysis by the patient collecting his or her own blood. This type of self-testing would not be possible with many other conventional sampling techniques.

Wicking action draws the blood into the absorbent tip. An optional barrier between the absorbent tip and the holder prevents blood from passing or wicking to the holder. The absorbent tip is composed of a material that wicks up substantially the same volume of fluid even when excess fluid is available (volumetric absorptive microsampling or VAMS™). The volume of the absorbent tip affects the volume of fluid absorbed. The size and shape of the absorbent tip may be varied to adjust the volume of absorbed blood and the rate of absorption. Volumes of various biological samples, including but not limited to blood, may be about 7-15 μL, about 20 μL and even up to about 30 μL. The sampling time may be about 2 seconds, about 3 seconds, about 5 seconds, or up to about 10 seconds.

In some embodiments, the material used for the absorbent tip is hydrophilic (e.g., polyester). Alternatively, the material may initially be hydrophobic and is subsequently treated to make it hydrophilic. Hydrophobic matrices may be rendered hydrophilic by a variety of known methods, such as plasma treatment or surfactant treatment (e.g., Tween-40 or Tween-80) of the matrix. In some embodiments, plasma treatment is used to render a hydrophobic material such as polyolefin, e.g., polyethylene, hydrophilic. Alternatively, the grafting of hydrophilic polymers to the surface and the chemical functionalization of active groups on the surface with polar or hydrophilic molecules such as sugars can be used to achieve a hydrophilic surface for the absorbent tip. Covalent modification could also be used to add polar or hydrophilic functional groups to the surface of absorbent tip. Other suitable materials for the absorbent tip include sintered glass, sintered steel, sintered ceramics, and sintered polymers of plastic, and sintered polyethylene.

In some embodiments, the microsampling device comprises an absorbent tip made of a hydrophilic polymeric material of sufficient size to absorb a maximum of about 20 μL of blood in about 2-5 seconds, and having a length of less than about 5 mm (0.2 inches) and a cross-sectional area of less than about 20 mm² and a density of less than about 4 g/cc. In some embodiments, the absorbent tips are composed of polyethylene and configured to absorb about 1-20 microliters of blood, preferably within 1-7 seconds, and more preferably within about 1-5 seconds. The absorbent tip may contain one or more of dried blood, dried anticoagulant or an internal standard.

In certain embodiments, the absorbent tips have a volume of about 35 mm³, absorb about 13-14 microliters of blood in about 3 seconds, absorb 9-10 microliters of blood in about 2.5 seconds, and have a pore volume of about 38%. In other embodiments, the absorbent tips have a volume of about 24 microliters, a density of about 0.6 g/cc, absorb about 10 microliters of blood in about 2.5 seconds, and have a pore volume of about 40%. In some embodiments, the volumetric absorptive microsampling device is a MITRA® tip, as described in US 2013/0116597, which is herein incorporated by reference in its entirety. The MITRA® Microsampling Device is a device for sample collection that is capable of accurately acquiring a small volume of blood (e.g., capillary blood) and storing it in a dried state. The device consists of a sampler body, which resembles a pipet tip, with an attached absorptive substrate or “absorbent tip” (MITRA® tip) that is designed to collect a fixed volume of 20 microliters (μL) of blood.

The absorbent tip may be shaped with an exterior resembling a truncated cone with a narrow and rounded distal end. In some embodiments, the holder has a cylindrical post that fits into a recess inside the center of the absorbent tip and extending along the longitudinal axis of the absorbent tip and holder. The conical shape of the absorbent tip helps wick the sample quickly and uniformly.

The holder may be adapted for use with a pipette. In some embodiments, a tubular, conical shaped holder is preferred, with the absorbent tip on the narrow end of the holder. The wider opposite end of the holder may be closed, or open and hollow, and may optionally be configured to attach to a pipette tip. The holder may have outwardly extending flanges that are arranged to abut mating structures in holders, drying racks or test equipment to help position the absorbent tip at desired locations in such holders, drying racks and test equipment.

In certain embodiments, the holder may include a pipette tip or a tapering, tubular structure configured to nest with a pipette tip. The absorbent tip may be composed of polyethylene, and both the absorbent tip and holder are made under aseptic conditions, or are terminally sterilized. The absorbent tip may contain dried anti-coagulant. In some embodiments, the holder has a plurality of ribs extending along a length of the holder. The ribs may have a height and length selected to keep the absorbent tip from contacting walls of a recess into which the holder and absorbent tip are placed for shipment, or for extraction of the dried blood in the absorbent tip.

After absorbing a small-volume sample, the absorbent tip is then dried. In some embodiments, the small-volume blood sample is dried for at least 10 minutes, at least 20 minutes, at least 30 minutes, at least 40 minutes, at least 50 minutes, at least 1 hour, at least 2 hours, at least 3 hours, at least 4 hours, at least 5 hours, at least 6 hours, at least 8 hours, at least 12 hours, at least 16 hours, at least 20 hours, at least 24 hours, at least 48 hours, at least 72 hours, or at least 96 hours at ambient or room temperature. In certain embodiments, the small-volume blood sample is dried for about 2-3 hours.

Drying can be done on a suitable rack or holder, or preferably the absorbent tip and holder can be transferred to a special drying container configured to facilitate drying while minimizing contact between the absorbent tip and the walls of the drying container or other potential contaminant surfaces. The drying container may have a desiccant to facilitate drying. The drying container may also provide a protective cover which may be sealed for transport to prevent contamination. In some embodiments, the cover has a surface onto which printed indicia may be written to identify the source of the dried blood sample and provide other relevant information. In some embodiments, the dimensions of the container, and the relative positions of the holders within the container, will conform to SBS Microwell plate specifications. The microsampling device and the drying container may be placed in a plastic bag along with a desiccant to assist with drying and can either be shipped in this fashion, or shipped after the desiccant is removed.

In some embodiments, the wider opposite end of the holder is hollow and the container has a first portion with a mounting projection portion sized to fit into and releasably engage the hollow end of the holder. Additionally or alternatively, the container has a second portion releasably fastened to the first portion and has a recess configured to enclose a portion of the holder for transportation of the holder. The container may comprise a plurality of openings allowing air to access the absorbent tip of the microsampling device. Moreover, the first portion may have a side with an access port therein of sufficient size and located so that indicia may be applied through the port and onto the holder when the holder is on the mounting projection.

Upon receipt at the testing location, the absorbent tip may be eluted in a predetermined volume of a suitable buffer (as described herein) either manually or via automated means to extract the nucleic acids or proteins of interest from dried blood. Physical agitation techniques such as sonication or vortexing of the fluid and/or the absorbent tip may accelerate the extraction process from the dried blood into a liquid sample matrix. Physical separation techniques such as centrifugation, evaporation/reconstitution, concentration, precipitation, liquid/liquid extraction, and solid phase extraction can be used to further simplify the sample matrix for further analysis.

Each container may enclose a plurality of holders, wherein each holder comprises an absorbent tip at its distal end and has a hollow proximal end. The container likewise has a plurality of elongated mounting projections each sized to fit into and releasably engage the hollow ends of the plurality of holders. The second portion of the container has recesses configured to separately enclose each of the plurality of holders in a separate enclosure within the container. In certain embodiments, each of the plurality of holders has a plurality of ribs extending along a length of the holder with the ribs configured to keep the absorbent tip from contacting walls of the container. As desired, a desiccant may be placed inside the container to help dry the blood in the absorbent tip or maintain dryness. Each holder may have visible indicia associating the holder with the container and with at least one other holder, such as serial numbers with various portions of the number indicating related holders/absorbent tips and the container in which the holders are shipped.

Methods of Determining HbA1c Fraction

Diabetes is exemplary of an indication that often requires frequent and routine screening and analysis of biological samples, and therefore could benefit from alternative sampling methodologies.

Disclosed herein is a method for determining the fraction of glycated hemoglobin (HbA1c) in a sample, comprising: (a) eluting a biological sample from a microsampling device used to collect the biological sample; (b) extracting hemoglobin from the biological sample; (c) measuring the concentration of HbA1c and the concentration of total hemoglobin (THb) in the sample; and (d) calculating the fraction of HbA1c in the THb.

Hemoglobin (Hb) consists of four protein chains with four heme portions, and is the red pigmented protein located in the erythrocytes. Its main function is the transport of oxygen and carbon dioxide in blood. Each Hb molecule is able to bind four oxygen molecules. Hb consists of a variety of sub-fractions and derivatives. Among this heterogeneous group of hemoglobins, HbA1c is one of the glycated hemoglobins, a sub-fraction formed by the attachment of various sugars to the Hb molecule. HbA1c is formed in two steps by the nonenzymatic reaction of glucose with the N-terminal amino group of the beta-chain of normal adult Hb (HbA). The first step is reversible and yields labile HbA1c. This slowly rearranges in the second reaction step to yield stable HbA1c.

In the erythrocytes, the relative amount of HbA converted to stable HbA1c increases with the average concentration of glucose in the blood. The conversion to stable HbA1c is limited by the erythrocyte's life span of approximately 100 to 120 days. As a result, HbA1c reflects the average blood glucose level during the preceding 2 to 3 months. HbA1c is thus suitable to monitor long-term glucose control in individuals with diabetes mellitus. More recent glucose levels have a greater influence on the HbA1c level.

The risk of diabetic complications, such as diabetic nephropathy and retinopathy increases with poor metabolic control. In accordance with its function as an indicator for the mean blood glucose level, HbA1c predicts the development of diabetic complications in Type 1 diabetes patients. Elevations of HbA1c are directly associated with the increased risk of these complications.

For routine clinical use, testing every 3 to 4 months is generally sufficient. In certain clinical situations, such as gestational diabetes, or after a major change in therapy, it may be useful to measure HbA1c in 2 to 4 week intervals. In some embodiments the biological sample is obtained from a patient having, or suspected of having diabetes, such as diabetes type 1 or type 2 or gestational diabetes.

In some embodiments, the biological sample is a dried fluid, such as dried serum, dried capillary blood, or dried whole blood. Other bodily fluids such as saliva, urine, and sweat may likewise be utilized in certain embodiments.

In some embodiments, the biological sample is collected from a patient via fingerstick, but venipuncture or other methods of obtaining a blood sample may be used as well.

In some embodiments, the biological sample is obtained and/or stored in a microsampling device (e.g., a MITRA® tip) which may comprise, among other components, an absorbent tip. In some embodiments, the biological sample is eluted from an absorbent tip of a microsampling device by incubating the absorbent tip in water.

In some embodiments, extraction comprises contacting the biological sample with a lysis buffer to lyse erythrocytes in the sample. After extraction of the hemoglobin from erythrocytes, the disclosed process may utilize an enzymatic method that specifically measures N-terminal fructosyl dipeptides of the (3-chain of HbA1c. For example, the enzymatic method may comprise a pretreatment process, the hemoglobin is transformed to methemoglobin by reaction with sodium nitrite. The addition of further reagents may result in the glycosylated N-terminal dipeptide (fructosyl-VH) of the (3-chain of hemoglobin being cleaved by the action of protease, and the hemoglobin may be transformed to a stable methemoglobin azide by contacting the methemoglobin with sodium azide. This allows for the concentration of total hemoglobin to be determined by measuring absorbance of the stable methemoglobin azide. Addition of a further reagent comprising fructosyl peptide oxidase (FPDX) may result in a reaction between the FPDX and the fructosyl-VH cleaved from the HbA1c in the sample. This allows for the concentration HbA1c to be determined by measuring the resultant hydrogen peroxide.

Accordingly, in some embodiments, the disclosed methods may further comprise contacting the sample with sodium nitrite and/or contacting the sample with one or more of a protease, sodium azide, and fructosyl peptideoxidase. In some embodiments, the concentration of THb is determined by measuring absorbance, and in some embodiments, the concentration of HbA1c is determined by measuring an indirect marker (e.g., hydrogen peroxide), but other detection methods known in the art, such as liquid chromatography (LC) and mass spectroscopy (MS), may be utilized in other embodiments. For example, some embodiments may employ tandem LC-MS/MS to detect the amount of HbA1c and THb in a given sample.

In some embodiments, the microsampling device may be a MITRA® tip, which is discussed in more detail above. In some embodiments, the microsampling device may hold a sample volume of no more than 10-20 μL.

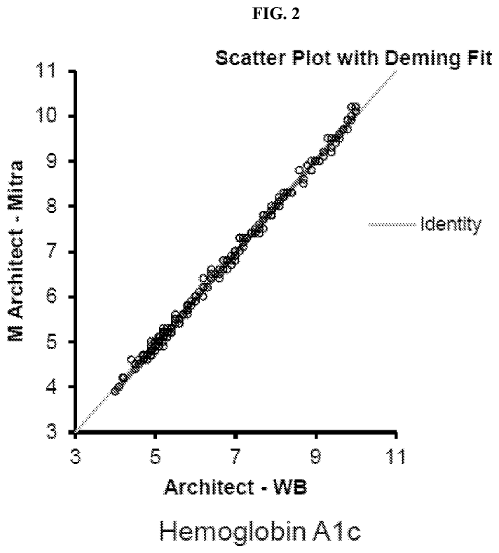

High fractions of HbA1c are indicative of diabetes or a lack of control of diabetes. As shown in FIG. 2, Deming regression analysis indicates that the HbA1c fraction is a validated marker of diabetic control. For example when HbA1c makes up 5.6% or greater of THb, it may be indicative of a lack of diabetic control, and the greater the percentage or fraction the more indicative of a lack of diabetic control (i.e., sustained high blood glucose levels). In some embodiments, when HbA1c is about 5.7%, 5.8%, 5.9%, 6.0%, 6.1%, 6.2%, 6.3%, 6.4%, 6.5%, 6.6%, 6.7%, 6.8%, 6.9%, or 7.0% or greater it is indicative of a lack of diabetic control.

The disclosed methods are further useful for streamlining diabetic testing and monitoring, which is critical for maintain the long term health of diabetic patients. Thus, some embodiments of the disclosed methods may comprise a patient self-collecting a biological sample (e.g., blood) via a microsampling device, mailing the sample to a pre-determined laboratory or testing facility, and then performing elution and extraction of the sample to measure and calculate the fraction of HbA1c to THb.

Additional Methods and Analytes

In addition to methods of detecting HbA1c and determining the fraction of HbA1c out of the total hemoglobin (THb) in a sample, the present disclosure provides methods for detecting other markers and analytes that may be useful in evaluating diabetic, metabolic, and cardiovascular health of an individual, as well as evaluating the overall health and nutrition of the individual. These methods comprise obtaining a biological sample from an individual using a microsampling device. The individual may have been diagnosed with, suspected of having, or is at risk of developing diabetes, metabolic disease, or cardiovascular disease.

Thus, the present disclosure further provides methods of detecting one or more analytes from a biological sample obtained with a microsampling device from the list consisting of HbA1c, total hemoglobin, glucose, low density lipoprotein particle number (LDLp), microalbumin, creatinine/estimated glomerular filtration rate (eGFR), thyroid stimulating hormone (TSH), C-reactive protein (CRP), vitamin D, omega 3, a marker of kidney function (e.g., creatinine, urea, uric acid, electrolytes), a marker of liver function (e.g., liver transaminases aspartate transaminase, alanine transaminase, bilirubin, albumin, alkaline phosphatase, gamma glutamyl transpeptidase), and a marker of thyroid function (e.g., TSH, T4, T3). In some embodiments, the detection and/or determination of the concentration of the analyte may comprise mass spectrometry, an immunological assay, an enzymatic assay, or an absorbance assay.

Various combinations of the disclosed analytes may be assessed in order to provide insight into an individual's diabetic, metabolic, cardiovascular, or overall health. For example, in some embodiments, HbA1c, glucose, LDLp, creatinine, and microalbumin may be assessed together to determine whether an individual's diabetes is under control. In some embodiments, TSH and CRP may be assessed together. In some embodiments, LDLp, HbA1c, and CRP may be assessed together to determine an individual's cardio-metabolic health. In some embodiments, LDLp, HbA1c, CRP, vitamin D, and omega 3 may be assessed together, along with, optionally, one or more markers of kidney, liver, and/or thyroid function to determine an individual's cardio-metabolic and nutritional health.

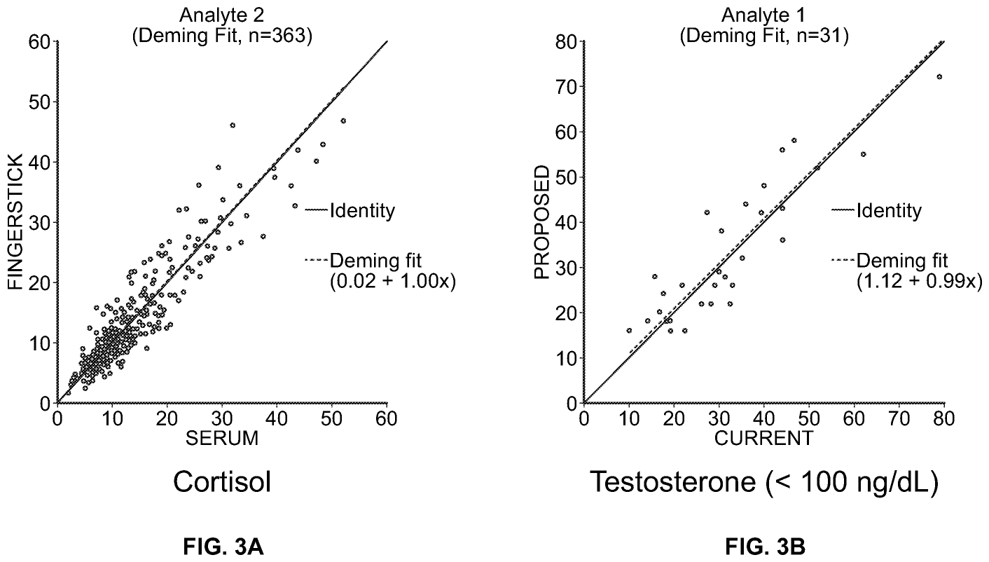

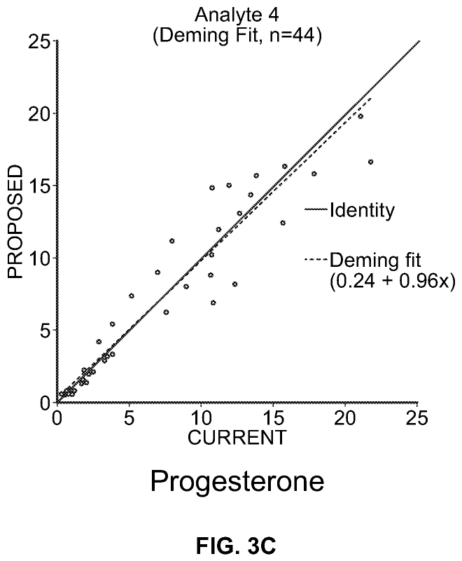

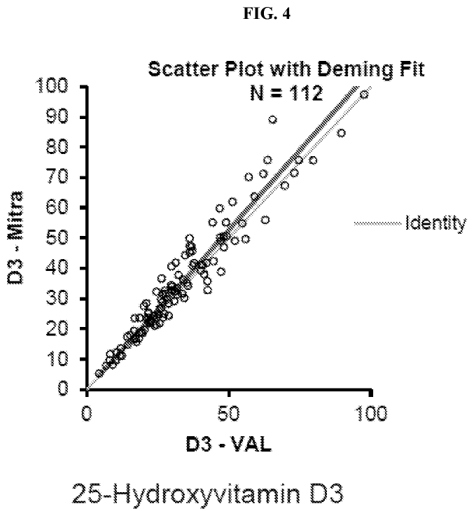

FIGS. 3 and 4 show that cortisol, testosterone, progesterone, and 25-Hydroxyvitamin D3 are detectable in biological samples when obtained via a microsampling device pursuant to the disclosed methods.

The methods illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred embodiments and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of determining a fraction of glycated hemoglobin (HbA1c) in a sample, comprising:
(a) eluting a biological sample from a microsampling device used to collect the biological sample an individual;
(b) extracting hemoglobin from the biological sample;
(c) measuring the concentration of HbA1c and the concentration of total hemoglobin (THb);
(d) calculating the fraction of HbA1c in the THb.

2. The method of claim 1, wherein the biological sample is obtained from an individual having or suspected of having diabetes.

3. The method of claim 1, wherein the biological sample is a dried fluid.

4. The method of claim 1, wherein the biological sample is dried serum, dried capillary blood, or dried whole blood.

5. The method of claim 1, wherein the biological sample was collected from a patient via fingerstick.

6. The method of claim 1, wherein the biological sample is eluted from an absorbent tip of the microsampling device by incubating the absorbent tip in water.

7. The method of claim 1, wherein extraction comprises contacting the biological sample with a lysis buffer to lyse erythrocytes in the sample.

8. The method of claim 7 further comprising contacting the sample with sodium nitrite.

9. The method of claim 7 further comprising contacting the sample with one or more of a protease, sodium azide, and fructosyl peptideoxidase.

10. The method of claim 1, wherein the concentration of THb is determined by measuring absorbance.

11. The method of claim 1, wherein the concentration of HbA1c is determined by measuring an indirect marker.

12. The method of claim 11, wherein the indirect marker is hydrogen peroxide.

13. The method of claim 1, wherein the THb or the HbA1c are measured by mass spectroscopy.

14. The method of claim 1, wherein the THb and the HbA1c are measured by mass spectroscopy.

15. The method of claim 1, wherein the microsampling device is a MITRA® tip.

16. The method of claim 1, wherein the sample volume of the microsampling device is no more than about 10 to about 20 μL.

17. The method of claim 1 further comprising detecting one or more of glucose, LDLp, creatinine, and microalbumin.

18. The method of claim 1, wherein the individual self-collects the biological sample.

19. The method of claim 18, wherein the individual shipped the microsampling device used to self-collect the biological sample to a testing facility in a pre-addressed envelope.

20. The method of claim 1, wherein a fraction of HbA1c/THb that is 6.5% or higher is indicative of a lack of diabetic control.

21. A method of detecting an analyte in a sample, comprising:
(a) eluting a biological sample from a microsampling device used to collect the biological sample from an individual;
(b) extracting two or more analytes from the biological sample;
(c) measuring the concentration of two or more analytes;
wherein the two or more analytes are selected from the group consisting of HbA1c, total hemoglobin, glucose, low density lipoprotein particle number (LDLp), microalbumin, creatinine/estimated glomerular filtration rate (eGFR), thyroid stimulating hormone (TSH), C-reactive protein (CRP), vitamin D, omega 3, a marker of kidney function (e.g., creatinine, urea, uric acid, electrolytes), a marker of liver function (e.g., liver transaminases aspartate transaminase, alanine transaminase, bilirubin, albumin, alkaline phosphatase, gamma glutamyl transpeptidase), and a marker of thyroid function (e.g., TSH, T4, T3).
